(19) United States
(12) Patent Application Publication
(10) Pub. No.: US 2006/0088893 A1
(43) Pub. Date: Apr. 27, 2006

(54) Prostate cancer therapeutics and diagnostics based on conformers of prostatic cid phosphatase

(76) Inventor: Vishwanath R. Lingappa, San Francisco, CA (US)

Correspondence Address: FOLEY & LARDNER LLP, 1530 PAGE MILL ROAD, PALO ALTO, CA, 94304, US

(21) Appl. No.: 10/512,025

(22) PCT Filed: Apr. 29, 2004
(86) PCT No.: PCT/US04/13457

§ 371 Date: Oct. 4, 2005

Related U.S. Application Data
(60) Provisional application No. 60/466,547, filed on Apr. 29, 2003.

(51) Int. Cl. A01K 67/027; G01N 33/574; C12P 21/06; A61K 39/00; C12N 9/16; C07K 16/40

(52) U.S. Cl. 435/7.23; 424/185.1; 435/196; 435/69.1; 435/320.1; 435/354; 800/18; 530/388.26

(57) ABSTRACT

The signals that determine important characteristics of prostate cancer, such as loss of dependence on androgens are described. A key protein, prostatic acid phosphatase, is shown to fold in different ways in response to cellular signals, and the conformer “mix” of this protein in prostatic cells may represent a fundamental difference between normal cells and cancer cells.

### FIELD OF THE INVENTION

The field of this invention is biological models and therapeutics for prostate cancer.

### BACKGROUND

Conceptually, cancer is a disorder of signaling pathways involved in cell growth and proliferation and the physiological controls over these processes, including a diverse array of apoptotic triggers and antiapoptotic factors. Many of these triggers and factors are secretory or membrane proteins which have either cleaved or uncleaved signal sequences. Cancer also can be affected, both positively and negatively, by a number of secreted growth factors and cytokines, which generally also have signal sequences. A host of data supports the notion that many of these factors and cytokines are multifunctional, or more precisely, are associated with both promoting and inhibiting particular signalling pathways. A present conundrum in the field is understanding how one factor can bring about one action at one point in time, and yet bring about a different action at another point in time.

The concentration of prostatic acid phosphatase (pacp) in blood is specific with respect to cancer of the prostate and in particular to the metastatic stage of the disease. However, this test is not entirely reliable because elevated levels of pacp can be observed in men with a non-cancerous disease of the prostate and pacp is not always seen in those with prostatic cancer. Pacp is a gene product found in both intracellular and secretory forms, which are identical in sequence and modifications (Lin M. F., et al. (1998) J Biol Chem. 273:5939-47). Loss of the intracellular form correlates with development of prostate cancer; conversion of pacp expression from an intracellular to a secretory form is associated with progression of prostate cancer from an androgen sensitive to an androgen resistant phase (Yeh S., et al. (1999) Proc Natl Acad Sci USA. 96(10):5458-63).

A biologically plausible mechanism connecting pacp to prostate cancer involves a demonstrated role for this protein in dephosphorylation of the Erb b oncogene and other tyrosine kinases, thereby downregulating signals for cell growth. Loss of this regulatory checkpoint on proliferation could be a key early step in the multi-step progression to malignant prostate cancer (Meng T. C. and Lin M. F. (1998) J Biol Chem. 273:22096-104). However, what has remained unclear from studies to date, is the molecular basis for the change in subcellular distribution of pacp from an intracellular to an extracellular location. Proteins that are retained in the ER in the absence of specific retention signals are typically viewed as “misfolded” molecules destined to be either refolded properly or degraded (Ellgaard L., et al. (1999) Science. 286:1882-1888). Yet, in the case of pacp, loss of intracellular localization, correlates with development and progression of prostate cancer.

The early events in biogenesis of secretory proteins have been extensively studied (see FIG. 1). The nascent polypeptide chain is targeted to the ER membrane by the signal sequence, a specialized channel is opened, and translocation to the ER lumen occurs by a cascade of interactions with receptors and other proteins (Stroud R. M. and Watler P. (1999) Curr. Opin. Struct. Biol. 9:754-759, Hegde R. S. and Lingappa V. R. (1997) Cell. 91(5):575-82). Concomitant with translocation, a wide range of co- and post-translational modifications of the nascent chain also occur, including glycosylation (Helenius A. and Aebi M. (2001) Science. 291(5512):2364-9), disulfide bond formation (Winter J., et al. (2002) J Biol Chem. 277(1):310-7), signal sequence cleavage (Kalies K. U. and Hartmann E. (1996) J Biol Chem. 271(7):3925-9) and acquisition of three-dimensional folded structure (Ellis R. J. and Hartl F. U. (1996) Faseb Journal. 10:20-6). What is far less clear is how these different events are interrelated and controlled, with some data suggesting that they are highly regulated (Rutkowski, D. T., et al. (2003) J. Biol. Chem. 278(32):30365-30372). The bioconformatic hypothesis has been proposed in part to explain this regulation (Lingappa V. R., et al. (2002) BioEssays. 24(8):741-9, see below).

Many of the early events in protein biogenesis can be recreated in cell-free transcription-linked translation systems (see FIG. 2). In at least three cases the cell-free system revealed unexpected features of protein biogenesis which, on further investigation, were found to occur in vivo using more sensitive means of detection than had been used previously (Lingappa J. R., et al. (1997) J Cell Biol. 136(3):567-75, Zimmerman C., et al. (2002) Nature. 415(6867):88-92, Lopez C. D., et al. (1990) Science. 248(4952):226-9, Bose H., et al. (2002) Nature. 417(6884):87-91). In effect, the cell-free system, by virtue of being slow and inefficient but of high fidelity, allowed those events to be more easily characterized.

Interaction with proteins termed molecular chaperones define a set of events termed “quality control” by which the newly synthesized protein is assessed and subsequently either allowed to leave the ER for export through the secretory pathway or is retained in the ER (Ellgaard L., et al. (1999) Science. 286:1882-1888). Retained proteins are either permanently localized to early compartments (e.g. if they have a ligand such as a C-terminal KDEL sequence) by virtue of recognition by membrane receptors that are recycled (Suokas M., et al. (2003) Biochem J. 370(Pt 3):913-20), or they are refolded and exported (Meng T. C. and Lin M. F. (1998) J Biol Chem. 273:22096-104), or they are retrotranslocated to the cytosol for degradation by the proteasome (Wiertz E. J., et al. (1996) Nature. 384(6608):432-8).

Conventionally it has been assumed that degraded proteins are, almost by definition, misfolded chains that failed attempts at refolding. Recently however, an example has been found where one form of the prion protein (PrP), that is normally degraded under one time/circumstance is rescued from degradation and exported through the secretory pathway to trigger apoptosis at another time/circumstance (Hegde R. S., et al. (1998) Science. 279: 827-834). Regulation of the fate of this form of PrP, termed CtmPrP, is distinct from that of another form termed SecPrP which is the only form observed in a normal adult brain. SecPrP trafficks through the secretory pathway efficiently at all times. Specific machinery of the ER is involved in regulating whether PrP is made as Ctm or Sec forms (Hegde R. S., et al. (1998) Molecular Cell. 2:85-9, Fons R. D., et al. (2003) J Cell Biol. 160(4):529-39). These two forms of PrP, differing only in 20 how they are folded, are termed conformers. These observations raise the possibility that protein degradation at the ER might not necessarily reflect misfolding. Rather, it might be due, in some cases, to the occurrence of alternate pathways of folding that are physiologically desirable under some circumstances but not others. In this case degradation might simply be a more effective means of regulation than control at the level of synthesis, for as yet unclear reasons.

The basis for loss of androgen sensitivity is a critical step in prostate cancer pathogenesis and remains poorly understood. It therefore is of interest to determine if it reflects a change in the conformer mix of pacp, for example, due to loss of pathways by which the mix of intracellular and secretory forms is regulated in trans. A switch from one to another conformer of prostatic acid phosphatase results in altered receptor interaction, signal transduction, half life, protein associations etc. could be central to one or more pathways of carcinogenesis and phenotypic variation in the development, immune surveillance, presentation and progression of prostatic cancer, or response to its treatment. By forcing a cell to make a conformer associated with stringent growth control establishes a novel means of anti-cancer therapy. In order to understand fully how conformers are made, the pathway has to be defined, compared and contrasted to that used by other conformers.

### SUMMARY OF THE INVENTION

Methods and compositions are provided for identifying one or more disease-related prostatic acid phosphatase and correlating the change from an intracellular to a secretory form with development and/or progression of prostate cancer. The method includes contacting a biological sample from a patient with prostate disease with a plurality of conformer-specific antibodies to pacp. Detection of the secretory conformer of pacp is indicative of malignant prostate cancer that is androgen insensitive; an increasing ratio of the secretory conformer as compared to the intracellular conformer is indicative of disease progression. Detection of only the intracellular conformer is indicative of a non-malignant prostate disease, and predominantly the intracellular conformer of prostatic cancer that retains androgen sensitivity.

Also provided are monoclonal antibodies that are substantially specific for the intracellular and secretory conformers of pacp, and hybridomas that produce the monoclonal antibodies, as well as isolated conformers that can be used for preparing the antibodies. The pacp conformers are produced by a method that includes the steps of swapping the native pacp signal sequence for a signal sequence with a known effect on protein folding, expressing the pacp in an in vitro translocation model system, employing modified lysates with microsomes to produce conformationally distinct pacp proteins, and isolating the resulting conformer. Knock-out and mutant small laboratory mammals also are provided for production of pacp antibodies, and elucidating mechanisms involved in regulating androgen sensitivity of prostatic cancer cells. The invention finds use in the development of means to force expression of one of more particular pacp conformer associated with androgen sensitivity in order to maintain androgen sensitivity of a prostatic tumor as a novel route for prostate cancer therapeutics, as well as diagnosis, prognosis and profiling of patients with prostatic disease.

### BRIEF DESCRIPTION OF THE DRAWINGS

FIG. 1. Shows the conventional view as compared to the bioconformatic view of translocation across the ER membrane. Conventionally (left), the only role of the signal sequence is targeting and translocation, and the ribosome membrane junction has been thought of as “closed”, and not subject to regulation. The bioconformatic hypothesis (right) proposes a different view of events in the ER (see below). According to that view, the precise pathway through the translocon determines the final conformational, topological or modification outcome. This hypothesis is supported by studies on biogenesis of several proteins including PrP (Hegde R. S., et al. (1998) Science. 279: 827-834, Hegde R. S., et al. (1998) Molecular Cell. 2:85-9, Fons R. D., et al. (2003) J Cell Biol. 160(4):529-39, Younes B., et al. (2001) J. Biol. Chem. 276(42):39145-9, Hegde, R. S., et al. (1999) Nature. 402:822-826), apo B (Hegde R. S., et al. (1996) Cell. 85(2):217-28), MDR1 (Skach, W. R., et al. (1993) J. Biol. Chem. 268:6903-6908), prolactin (Rutkowski, D. T., et al. (2003) J. Biol. Chem. 278(32):30365-30372), and most recently, pacp (see preliminary studies). Thus, the role of the ER is not just to get proteins into the secretory pathway but to regulate their conformation. However this aspect is extremely difficult to study in the absence of tools such as conformation-specific monoclonal antibodies, etc. Hence it has gone generally unappreciated and may explain numerous complex biological events including many relevant to prostate cancer (Lin M. F., et al. (1998) J Biol Chem. 273:5939-47, Yeh S., et al. (1999) Proc Natl Acad Sci USA. 96(10):5458-63, Meng T. C. and Lin M. F. (1998) J Biol Chem. 273:22096-104).

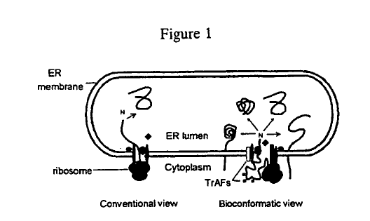

FIG. 2. Shows a cartoon depicting the steps in the Cell-Free Translation System (CFTS). Eukaryotic cells (bottom), classically rabbit reticulocytes (Perara E., et al. (1986) Science. 232(4748):348-52) and wheat germ (Jamieson J. D. and Palade G. E. (1968) J Cell Biol. 39(3):589-603), are homogenized and cell-free extracts are prepared. Heterologous microsomal membrane vesicles are also prepared (Hegde R. S., et al. (1996) Cell. 85(2):217-28). Translation of added mRNA is carried out under various conditions as previously described (Hegde R. S., et al. (1996) Cell. 85(2):217-28). The advantage of this method over events studied in vivo (e.g. by pulse chase analysis of cultured mammalian cells) is that this method is extremely slow and inefficient yet highly faithful to events in vivo. As a result, events, intermediates and steps not readily detectable in vivo can be detected in the cell-free system (see text). Manipulations in cis that allow events to be analysed in CFTS include truncation of mRNAs (Perara E., et al. (1986) Science. 232(4748):348-52), manipulations in trans include addition of antibodies (Hegde R. S., et al. (1996) Cell. 85(2):217-28), fractionation and reconstitution of the cytosol (Bose H., et al. (2002) Nature. 417(6884):87-91) or the membrane (Hegde R. S., et al. (1998) Molecular Cell. 2:85-9).

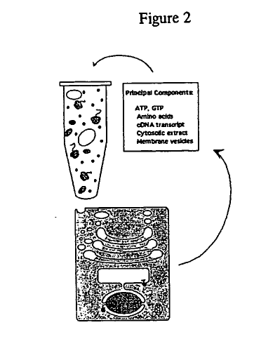

FIG. 3 is a cartoon depiction of protein folding in various environments. FIG. 3A shows that the precise folding/modification pathway taken by a chain will depend on the precise environment it sees including the precise protein-protein interactions it undergoes. This could be different for particular subpopulations of chains resulting in the potential for heterogeneity as indicated at the bottom. FIG. 3B shows the consequences of regulation of various parameters. Changes in organization of the translocon (see Rutkowski, D. T., et al. (2003) J. Biol. Chem. 278(32):30365-30372), in the r-mj (see Rutkowski D. T., et al. (2001) Proc. of the Nat. Acad. of Sci. of the U.S.A. 98:7823-7828), and in the membrane (see refs Hegde R. S., et al. (1998) Molecular Cell. 2:85-9 and Fons R. D., et al. (2003) J Cell Biol. 160(4):529-39) are known to alter conformation of various proteins. Such regulation alters pacp biogenesis in ways relevant to prostate cancer.

FIG. 4 shows the results of expression of pacp (which has three N-linked glycosylation acceptor sites at amino acid residues 94, 220, and 333) in the following situations: engineered behind an SP6 promoter either directly (wild type (wt) pacp) or after replacing the N and H domains of its signal sequence with those of prolactin (prl-pacp), growth hormone (gh-pacp), or IgG (IgG-pacp). The constructs are expressed by transcription-linked translation in a cell-free system either without added microsomal membranes (−), with microsomal membranes (+), or with membranes and acceptor peptide (AP, a tripeptide competitive inhibitor of N-linked glycosylation that abolishes all N-linked glycosylation at the concentrations used), for 45 min. Samples were analysed by SDS-PAGE and AR (see FIG. 2). The arrowhead indicates the position of signal cleaved, non-glycosylated pacp; the arrow with short tail, indicates the position of precursor which comigrates with singly glycosylated pacp; the arrow with medium-sized tail, indicates the signal cleaved and doubly glycosylated species (shifted up because addition of N-linked carbohydrate at two of three sites more than offsets shift down due to signal sequence cleavage); the arrow with the long tail, indicates doubly glycosylated species with inefficient signal sequence cleavage, a phenomenon observed in the cell-free system at early time points but not with longer incubation (see FIG. 5).

FIG. 5 shows the results when site directed mutagenesis was used to change individual N residues of glycosylation acceptor sites to Q and thereby abolishing their ability to serve as N-linked glycosylation acceptors. The numerals 1, 2, 3 refer to the sites abolished at amino acid residues 94, 220 and 333 from the N terminus of this 386 amino acid residue polypeptide. Plasmids were then expressed as in FIG. 4. Only reactions+microsomal membranes are shown. Because of long incubation time (90 minutes) signal sequence uncleaved and doubly glycosylated chains are not observed for gh and IgG signal sequences as in FIG. 4.

### DESCRIPTION OF THE SPECIFIC EMBODIMENTS

Methods and compositions are provided for elucidating the mechanisms relating to the signals required to maintain androgen sensitivity of prostatic cancer, in particular the role of conformers of pacp. A protein translocating system is employed in determining the components of the system and their function, that modulate folding of pacp using chimeric genes employing an unnatural signal sequence to provide “conformers.” Conformers are proteins that have at least substantially the same amino acid sequence as each other, but have different physical topology or topography. By topology is intended the different placement of the protein, e.g. C-cytosolic as compared to N-cytosolic. By topography is intended change in external conformation or shape. Topography as used herein refers to proteins of substantially the same or similar amino acid sequence but having different three dimensional shapes due to differences in folding/conformation. Proteins of substantially the same amino acid sequence include those with conservative amino acid substitutions (i.e. a small or large side chain for a small or large side chain, respectively; or an acidic, basic, polar or hydrophobic side chain for an acidic, basic, polar or hydrophobic side chain, respectively), which changes do not alter the protein conformation or topology.

Methods for identifying the multiple pacp gene products that regulate signal sequence mediated selection of folding funnels during protein biogenesis also are provided, using a reticulocyte lysate fractionation scheme. Also provided are methods for identifying conformers, providing in vitro and in vivo systems for these purposes, and identifying compositions that modulate pacp conformation during translocation.

The methodology for fractionation of translation extracts and fractionation of solubilized membranes with reconstitution of subfractions containing or missing particular trans-acting activities, offers several advantages over existing systems.

For the cell-free protein translation mixture, various systems may be employed (Erickson and Blobel, Methods Enzymol. 96:38-50 (1983); Merrick, Methods Enzymol 101:606-615 (1983)). Illustrative are wheat germ extract and rabbit reticulocyte extract, available from commercial suppliers, e.g. Promega (Madison, Wis.), as well as high-speed supernatants thereof. Other references include U.S. Pat. Nos. 5,998,163; 5,998,136 and 5,989,833. Such systems include, in addition to the cell-free extract containing translation machinery (tRNA, ribosomes, etc.), an energy source (ATP, GTP), and a full complement of amino acids. Methods known in the art are used to maintain energy levels sufficient to maintain protein synthesis, for example, by adding additional nucleotide energy sources during the reaction or by adding an alternative energy source, e.g., creatine phosphate/creatine phosphokinase. The ATP and GTP present in the standard translation mixture will generally be at a concentration in the range of about 0.1 to 10 mM, more usually 0.5 to 2 mM. Generally, the amount of the nucleotides will be sufficient to provide at least about 5 picomolar of product, preferably at least about 10 picomolar of product. The process can be readily expanded to a large scale; the materials required for the fractionation are either inexpensive (common chemicals) or reuseable (centrifuge tubes, ion exchange resin); and the final products have a long shelf life when stored appropriately. This system can be readily adapted to other translation systems such as the wheat germ translation system. In some cases, the fractionated system results in an enormous increase in the translation efficiency (see above). Furthermore, the ability to mix and match components from multiple such fractionated systems allows tissue specific events involved in the biogenesis of certain proteins, such as pacp, to be studied. This is potentially useful for the identification of factors, by complementation, involved in such tissue specific events. Examples of tissue specific differences have been documented (Wolin and Walter, J. Cell Biol (1989) 109:2617-2622; Lopez et al., Science (1990) 248:226-229).

Numerous signal sequences have been identified from different proteins and appear to be capable of operating conjugated to a broad range of unnatural proteins. The signal sequences are usually N-terminal, but may be internal to the protein or C-terminal. Signal sequences are selected from proteins that are known to have a specific mechanism for translocation affecting the conformation of the product or may be synthetic, where the translocational effect is known or determined. It is now known that the signal sequence affects the conformation of the protein that is translated in conjunction with the translocon. Without being bound by any theory, it is hypothesized that the signal sequence directs whether the ribosome forms a tight, loose or intermediate junction with the endoplasmic reticulum (ER) and the selection of the channel and accompanying processing proteins through which the translated protein is translocated and processed. For example, replacing a signal sequence of a protein with a signal sequence from preprolactin results in a tight junction, while the signal sequence from pre-β-lactamase provides a loose junction. Proteins which provide tight junctions include:growth hormone; and loose junctions include: immunological heavy chain and yeast alpha-factor; and intermediate junctions include ductin, calreticulin, PrP, angiotensinogen and MDR-1, where the division between the different conformers may be attributed to a variety of mechanisms. (See, for discussion, Hegde and Lingappa, (1996) Cell 85, 217-228 for a discussion of the effect of the ribosome-membrane junction.) For a general discussion of the mechanism of translocation, see, for example, Ellgaard, et al., (1999) Science 286, 1882-1888; Wickner, et al., (1999) Science 286, 1888-1893; and Ibba and Soll, (1999) Science 286, 1893-1897.

Signal sequences may be rated by using a lysate competent for expression, microsomes and proteinase K. The degree of proteolysis occurring with different signal sequences and a common gene is indicative of the nature of the junction of the ribosome with the ER. By using signal sequences having different degrees of junction tightness, the conformation of the resulting pacp can be modified. These different conformers may be used in a number of ways. The conformers may be used for the production of antibodies, either antisera or preferably monoclonal antibodies. The antisera and antibodies are prepared in conventional ways using the different pacp conformers to immunize a mammalian host, usually a rodent such as a mouse in the case of monoclonal antibodies, with or without an adjuvant, followed by additional injections of the protein at biweekly or longer intervals and monitoring the level of antisera. For monoclonal antibodies, splenocytes may be isolated, immortalized and screened. Those hybridomas which produce antisera which can distinguish between the conformers are expanded. A library is produced of antibodies that distinguish between two or more pacp conformers. The antibodies may then be used to isolate each of the conformers and assay for the different conformers in hosts, particularly human hosts, using physiological samples appropriate to the nature of the protein.

Signal sequences may also be rated by analysis of crosslinking patterns generated when truncated transcripts encoding those signal sequences at the 5′ end of the authentic coding region of pacp are expressed by cell-free translation and subject to chemical crosslinking including Lys and Cys specific cleavable and uncleavable crosslinkers, with analysis of the crosslink patterns by immunoprecipitation and polyacrylamide gel electrophoresis in sodium dodecyl sulfate and subsequent autoradiography.

Different pacp conformers are screened by employing matrices of different oligopeptides and/or oligonucleotides. See, for example, U.S. Pat. Nos. 5,631,734; 5,856,102 and 5,919,523. These matrices are available commercially and can be prepared in relation to a particular binding pattern. A physiological sample is added and which of the conformers are present and the amount of each is determined. The matrix also can be used to isolate particular pacp conformers by their binding affinity to the matrix. The matrix and antibodies used together in the same assay can be used in conjunction as confirmation of the results of each, and to isolate a particular pacp conformer. In assays by themselves or in conjunction with other affinity binding assays, the antibodies may be labeled with a detectable label, e.g. fluorescer, luminescer, phosphorescer, enzyme, radioisotope, and the like. Numerous protocols are available for defining specific epitopes, by which the pacp conformers may be distinguished. In some instances, it may be desirable to use two or more antibodies, where the conformer may be defined by steric inhibition of binding, different affinity constants, or the like.

By virtue of the fact that the conformers can be distinguished by oligomers, particularly of oligopeptides, these oligopeptides may serve for identifying the different conformers. The oligopeptides can be used in competitive assays for identifying other oligopeptides which compete for the site or other compounds, particularly small organic compounds, natural or synthetic, of less than 5 kDal, usually less than about 2.5 kDal, which bind to the conformer. These compounds may then be used in turn to identify other compounds having greater affinity for the site. In this way drugs may be identified that are specific for one conformer, as compared to other conformers. The various binding entities may be used in assays, where the entity may be labeled to identify binding to the conformer. The assays may be homogeneous or heterogeneous.

If desired, random mutations of the wild-type and/or chimeric gene can be used to express the resulting gene. The protein may be analyzed for similarities and differences with the parent gene. The effect of the mutation may be to change the conformer from one conformation to another or to change an epitope, as determined by antibody binding. Alternatively, the mutation may generate a pacp with differences in crosslinking pattern or conformation as scored by altered reactivity to chemical modifying agents under non-denaturing conditions, including N-sulfobiotin, trinitrobenzenesulfonic acid (TNBS), N-ethyl maleimide (NEM). Pacp genes, and the proteins which they express, from human patients or animals, where a mutation is naturally occurring or a result of mutating the gene, may be compared with the randomly mutated gene and its expression product in a mammalian host. In this way, loss of androgen sensitivity for example, and/or change from an intracellular to a secretory conformer of pacp resulting from such mutations may be diagnosed and treatments developed.

The pacp conformers can be used for biomedical purposes in aiding in the diagnosis and treatment of patients. The pacp conformers serve to differentiate between individual hosts, who produce the conformers in different ratios, normally or under different environments. One of the environments is the presence of a compound, particularly a drug, and the binding affinity and/or modulation of activity of a target physiologically active compound, usually a protein, such as a membrane receptor, channel, enzyme, transcription factor, housekeeping protein, cytoskeletal protein, membrane protein, and the like. The conformers may be mobile or immobile proteins, being bound to membranes or free in solution. A compound would be mixed with the different conformers, in the same or different vessel, and the binding of the compound to the conformer determined. The determination may be made in a variety of ways, either competitive or non-competitive. The amount of compound remaining in the solution may be determined, where a reduction in amount would be indicative of the binding of the compound to the conformer. Alternatively, a labeled compound which binds to the conformer may be used, where interest in binding is as to a particular region of the protein, such as in the case of enzymes and receptors, as well as other proteins where particular sites are associated with biological activity, e.g. G-proteins, transcription factors, DNA binding proteins, and the like.

The pacp conformers are also useful in determining differences in activity in relation to their physiological activity. The binding affinity for their binding partners can be determined in assays, either homogeneous or non-homogeneous, which allows for evaluation of the level of activity of a mammalian host, such as androgen sensitivity of prostate cancer cells as compared to normal prostate cells, preferably from the same host, in relation to the proportion of the two conformers. Again, numerous protocols are available for detecting binding between a ligand and a receptor or two or more proteins involved in complex formation. The two proteins may be labeled with a fluorescer and an energy receptor, so that binding of the two proteins together would reduce the level of emission at the wavelength of the fluorescer and increase the emission at the level of the energy acceptor. Alternatively, one of the proteins can be bound to a surface and the level of binding of the other protein to the bound protein determined by labelling the second protein. Alternatively, each of the proteins can be bound to different particles, where one particle produces a compound, which activates the other particle, such as LOCI. Where the labeled entity is small, such as an oligomer or small organic molecule, a fluorescer may be used as the label and fluorescence polarization employed for detection. Illustrative of assays are the assays described in U.S. Pat. Nos. 5,989,921; 4,806,488; 4,318,707; 4,255,329; 4,233,402; and 4,199,559.

In determining the presence of conformers when screening physiological samples, the samples may be any sample where pacp is found, generally blood or cells, particularly prostatic cells. The sample may be subject to pretreatment, such as adding citrate to blood, coagulating and separating erythrocytes, dilution, extraction, etc. Thus, the pacp conformers can be identified as being associated with particular indications, which may relate to loss of hormone sensitivity, disease progression, response to drugs, cellular degeneration, apoptosis, etc.

Illustrative of the power of the subject invention is the investigation of prions. It was found that by varying the signal sequence the native conformation ^{sec}PrP, and two other conformations, ^{Ntm}Prp and the neurodegenerative ^{Ctm}Prp, could be obtained in varying amounts. This aids in the determination of the mechanism of the change in the proportion of formation of the neurodegenerative form as compared to the wild-type form. In an analogous manner, the signal sequences for pacp can be varied and then whether the protein can be produced in varying conformations can be determined. If different conformers are found, as described above, the presence of the different conformers is established in prostates from healthy normal patients as compared to patients who have prostatic cancer which is androgen dependent, androgen sensitive, or androgen independent or which contain mixtures of dependent and/or sensitive and/or independent cells. Drugs can then be screened as described above against the undesired conformer(s).

By using different signal sequences the basis for the change in proportion between a desired conformer and an undesired conformer can be investigated. In conjunction with, in addition to or in place of, one can use in vitro lysates for investigating the role of ER associated proteins with the formation of the pacp conformers. Microsomes are prepared lacking all but the essential proteins for translocation. The individual proteins then are replaced individually or in combination to determine the effect of the presence of the protein(s) on the formation of the various pacp conformers. Once the protein(s) involved in the formation of the conformers is determined, healthy and abnormal patients can be screened for the presence of the protein(s) involved in the formation of the various conformers and to determine the presence of mutations or different conformers. In this manner, not only can the mechanism by which pacp conformers fold in the translocon be elucidated, but new targets for therapies can be established also.

By varying the signal sequence of pacp, genes for expression in mammalian hosts can be obtained. Methods of replacing a DNA sequence to provide a chimeric gene are legion today. See, for example, Sambrook, et al. (1989), A Laboratory Manual, Second edition, Cold Spring Harbor Press. Briefly, the DNA for the gene is isolated knowing the amino acid sequence and using degenerate probes. Once the gene is produced it may be introduced into one of numerous commercially available vectors and cloned and/or an expression vector may be employed having a transcriptional regulatory region 5′ of the sense strand to provide for expression. By using mammalian cells, the conformers from the different constructs can be produced and isolated and assayed as described above. In this way, significant amounts of the different conformers can be isolated.

In an initial stage, the conformers can be concentrated using different separation techniques, such as HPLC, capillary electrophoresis, affinity chromatography, and the likee. The initial separation may serve solely to enhance the concentration of the conformers in a particular fraction or may provide for separation of the conformers. In the former case, it may be necessary to further purify the conformers, using techniques known to those of skill in the art. Various separation media may be employed, involving ion exchange, sieving media, affinity media, etc., using different eluents to establish procedures for separation and isolation of the different conformers. Various procedures have been developed and each set of conformers will use protocols that optimize the separation with minimum denaturation. These protocols may be readily identified by those of skill in the art of protein purification. The purified pacp conformers may then be used for assays, for x-ray crystallography, to identify differences in three-dimensional structure, the amino acids associated with the different epitopes, the interactions with proteins with which the various conformers are associated, as well as other proteins to which the pacp conformers may bind, and the like.

Following the procedures described above and in the Examples or other functionally equivalent procedures, the subject invention allows for investigation of the loss of androgen sensitivity in prostate cancer. By preparing expression constructs for expression of the various conformers of pacp, one can modify cells and animals to produce different forms of pacp. In this way, the effect of the addition of various candidate agents for treating prostate cancer can be studied in culture and in vivo and the change in the formation of the different conformers in the presence of the agents in culture and in vivo can also be determined. The controlled presence of the different pacp conformers in combination with a study of the etiology of prostate cancer can be used to determine whether other entities are involved in the etiology of the disease, by doing expression analysis of the effect of the different conformers on expression in the same and different cells and on the physiology of the host. Animal models of prostate cancer with varying depencies on androgens can be developed with the various chimeric constructs in the presence or absence of the native pacp gene, since the host gene may be knocked-out and replaced with a chimeric gene. Alternatively, mutations can be introduced into the pacp gene that result in a modification of folding and a modification in the dependence on or interaction with proteins associated with androgen dependence.

The following examples are offered by way of illustration not limitation.

### EXAMPLES

Abbreviations

Prostatic acid phosphatase (pacp); endoplasmic reticulum (ER), ribosome-membrane junction (r-mj); polyacrylamide gel electrophoresis in sodium dodecyl sulfate (SDS-PAGE); Prion protein (PrP); transmembrane form of PrP with C-terminus in the ER lumen (CtmPrP); fully translocated form of PrP (SecPrP). Autoradiography (AR).

General Methods

Methods relating to the analysis of the topology of mutant PrP molecules at the ER membrane with cell-free translation as well as production, histological and biochemical characterization of mice expressing mutant PrP transgenes are provided in Hegde et al, Science (1998) 279: 827-834, which disclosure is incorporated herein by reference. Methods relating to substrate-specific regulation of the ribosome-translocon junction by N-terminal signal sequences as well as plamid constructions for use in the methods are provided in Rutkowski et al, Proc. Nat. Acad. Sci USA (2001) 98: 7823-7828, which disclosure is hereby incorporated by reference. See also Lingappa et al, BioEssays (2002) 741-748, which disclosure is hereby incorporated by reference.

### Example 1

### Evidence for Conformers of pacp

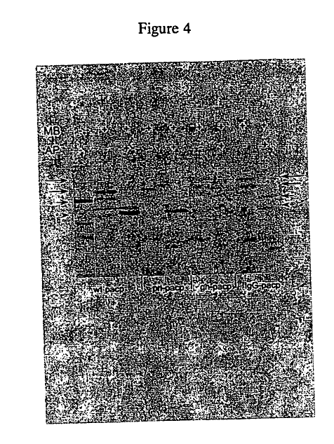

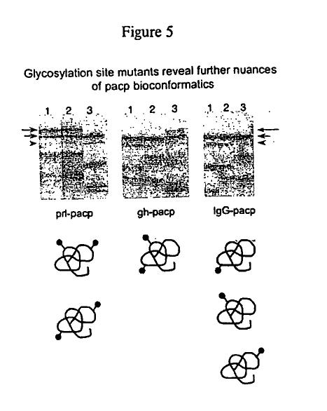

The purpose of this study was to determine whether conformers of pacp can be produced. There are at least two different ways in which conformer choice is regulated during protein biogenesis. First, PrP was found to utilize open versus closed r-mjs to achieve its alternate conformational fates (that happen to differ also in topology thereby making them relatively easier to detect). Second, it was found that mutations of the prolactin signal sequence which had no effect on targeting or translocation per se were able to redirect prolactin biogenesis from one conformer to another (as assessed by utilization of an engineered glycosylation site) without affecting the nature of the ribosome-membrane junction (Rutkowski, D. T., et al. (2003) J. Biol. Chem. 278(32):30365-30372). The N and H domains of the signal sequence were found to be sufficient to confer these features (Rutkowski, D. T., et al. (2003) J. Biol. Chem. 278(32):30365-30372). In an attempt to extend these findings to pacp, we swapped the N and H domains of the signal sequence of pacp to those of three signal sequences known to be regulated in different ways (i.e. one open and two closed r-mjs, see Rutkowski, D. T., et al. (2003) J. Biol. Chem. 278(32):30365-30372). The pattern of glycosylation of pacp (which has three N-linked glycosylation acceptor sites) was found to be similar in that pacp behind all tested signal sequences was predominantly doubly glycosylated (see FIG. 4). One difference was that the two signal sequences favoring glycosylation in the prolactin reporter system (Rutkowski, D. T., et al. (2003) J. Biol. Chem. 278(32):30365-30372) were found to have delayed signal sequence cleavage, consistent with previous observations. This was however, a kinetic effect as efficient signal sequence cleavage was observed on longer incubation (see FIG. 5).

Despite similar overall glycosylation of pacp behind different signal sequences, dramatic differences were found in the pattern of glycosylation site used as a function of signal sequence swapping (see FIG. 5). The significance of these observations is two fold. First, it demonstrates that different classes of signal sequences can redirect pacp biogenesis to different pathways. Second it demonstrates that profound effects on glycosylation site utilization, likely reflecting differences in pacp folding, are remarkably hard to detect, and are apparent only when extraordinary lengths are taken as in FIG. 5. Taken together, these findings strongly suggest that pacp behind different signal sequences is synthesized as different conformers, providing plausibility to the theory of the invention that different conformers account for differences in subcellular trafficking and function of pacp with respect to prostate cancer (1)(2)(3). The results shown in FIG. 6 suggest that pacp is made in multiple conformers), and that these conformers correlate with the subcellular location of pacp. According to this theory, forced expression of one conformer, but not of another, would confer androgen sensitivity to androgen-insenstive prostate cancer cell lines.

### Example 2

### Evaluation of Trafficking of pacp Synthesized Behind Different Signal Sequences

The purpose of this study is to determine if pacp synthesized behind different signal sequences and shown (see Example 1 above) to display different patterns of glycosylation, displays differences in trafficking (e.g. is retained in an intracellular compartment as opposed to being secreted). If so, this would be strong support that the different conformers defined by glycosylation pattern are relevant to the provocative intracellular distribution difference in pacp in normal prostate cells as compared to some transformed prostate cancer cell lines.

Signal sequences have been defined as being of different classes based on the ability of some of them to redirect the biogenesis of several unrelated proteins in a distinctive fashion, even though all of them are equally and fully competent to achieve translocation to the ER lumen. The distinctive features specific for a given class of signal sequence all relate to conformation, namely intrinsic protease resistance (Hegde R. S., et al. (1998) Science. 279: 827-834), and glycosylation state (Rutkowski, D. T., el al. (2003) J. Biol. Chem. 278(32):30365-30372) and pattern (FIG. 4/5).

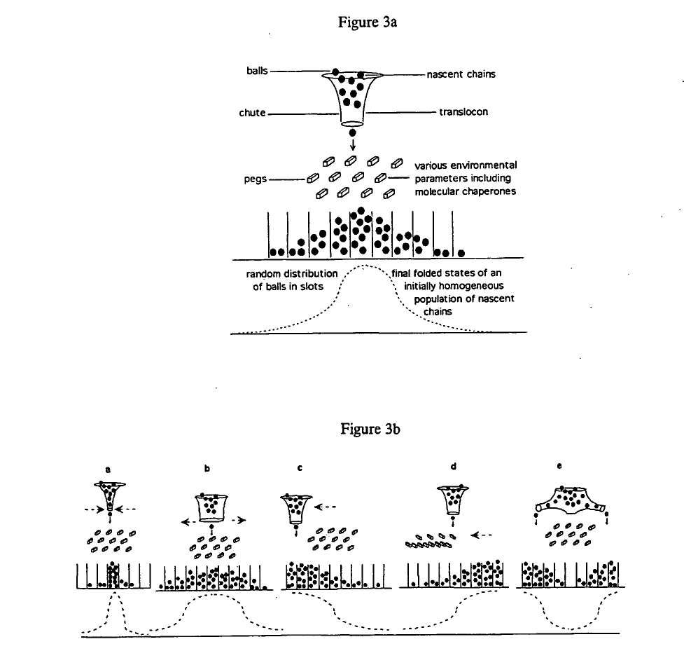

In carrying out this aim the same constructs that were expressed by cell-free transcription linked translation in FIG. 3 are engineered into mammalian cell transfection vectors behind the CMV promoter. These constructs are then expressed in various cell lines including LNCAP C-81 prostate cancer cells that are known not to express pacp and to be androgen-insensitive. After confirming expression by western blot of cell lysates, cells are analysed 24, 48 and 96 hrs after transfection to determine the steady state distribution between pacp in the medium, whole cell lysates, and fractionated extracts. Then the same experiments are performed with a radioactive methionine pulse of 10 to 30 minutes followed by a non-radioactive methionine chase of 6, 12, 24 and 48 hrs, with quantitation of newly synthesized pacp distribution over time. Cell fractionation is performed at each time point and the location of pacp determined by solution immunoprecipitation, SDS-PAGE and autoradiography. Parallel experiments are performed in C-33 cells which express pacp and are androgen-sensitive.

Since pacp made behind each of four distinct classes of signal sequences show different patterns of glycosylation, it is a theory of the invention that they present different shapes to proteins with which they interact, either intrinsically apart from their glycoform differences, or solely as a consequence of those differences. The former is more likely by analogy to prion protein where the distinction was first made, and where intrinsic protease resistance allows different conformers to be scored in the absence of glycosylation. Thus it is theorized that at least one of the four forms of pacp made will be distinct from the others in trafficking and will mimic the intracellular vs extracellular dichotomy on the basis of which a connection to prostate cancer has been made. Such differences in trafficking are readily detected by the pulse-chase technique and fractionation through differential ultracentrifugation studies described above which represent a classical approach to analysis of intracellular trafficking through subcellular compartments (Jamieson J. D. and Palade G. E. (1968) J Cell Biol. 39(3):589-603).

To establish that the conformers generated in the cell-free system are true to what is made in vivo, since mutants in which individual glycosylation sites were abolished were used to establish that pacp made behind different signal sequences is differentially glycosylated, conformer specific monoclonal antibodies are used: this is a simple, efficient and quick means of scoring conformational differences by differential reactivity to a panel of conformation-specific mAbs. Alternatively, each clone can be expressed by mammalian cell transfection to show, as has been the case for conformers of PrP studied previously, that the cell-free system is faithful, albeit inefficient, when compared to the results from studies in vivo.

### Example 3

### Evaluation of Whether One of More Individual pacp Conformers are Responpsible for Conferring Androgen Dependence on Prostate Cancer Cells

The purpose of these experiments is to determine if some but not other pacp conformers (defined either by glycosylation pattern as described in Example 1 above or by intracellular distribution as described in Example 2, above) are able to entrain, in an androgen-dependent manner, the growth of a prostate cancer cell line that displays androgen independence in the absence of pacp expression, i.e. will some but not all pacp conformers have the same or at least a similar effect to that observed with wild-type pacp transfection. Wild-type pacp transfection into a LNCAP C-81 prostate cancer cell line renders the cell line androgen-sensitive. Wild-type pacp, by analogy to proteins studied previously (Hegde R. S., et al. (1998) Science. 279: 827-834, Rutkowski, D. T., et al. (2003) J. Biol. Chem. 278(32):30365-30372), represents a heterogeneous mix of conformers regulated in trans. Since multiple pacp conformers have been demonstrated based on differences in glycosylation, these forms could differ in the extent to which they entrain LNCAP C81 cells.

LNCAP C-81 cells are transfected with the same pcDNA contructs indicated above and the phenotype of the cells observed for each pcDNA construct in the absence and/or presence of androgen or in the presence of an androgen antagonist (1)(2)(3). In particular DNA synthesis, (measured by tritiated thymidine incorporation), cell morphology, and various measures of apoptosis including caspase activation and PARP cleavage is assayed.

### Example 4

### Determination of the Mechanism by Which pacp Glycosylation Pattern is Altered by the Signal Sequence Utilized

The purpose of these experiments is to determine the mechanism by which pacp glycosylation pattern is altered by a change in the class of signal sequence utilized.

First, the fidelity of pacp signal sequence cleavage is confirmed by Edman degradation of cell-free translation products synthesized in the presence of microsomal membranes derived from the ER. Since the cleavage products from several different sized signal sequences comigrate on high resolution SDS PAGE, and since only the N and H domains of the signal sequences are swapped to generate the various constructs, it is highly likely that the cleavage is correct, but this critical point needs to be experimentally verified.

Second, the nature of the ribosome-membrane junction is assessed by which pacp conformers directed by different signal sequences are made. Some r-mj's have been found to be “open” while others are “closed” (Rutkowski D. T., et al. (2001) Proc. of the Nat. Acad. of Sci. of the U.S.A. 98:7823-7828; see appendices #2 and #3). Differences in r-mj account for important dimensions of pathway and therefore conformer choice in biogenesis of some but not all (Rutkowski, D. T., et al. (2003) J. Biol. Chem. 278(32):30365-30372) of other proteins studied. These studies are used to determine whether pacp represents a new form of bioconformatic regulation or simply a variation on a previously defined theme.

Finally, chemical crosslinking is carried out with Lys and Cys-reactive reagents (e.g. DSS and BMPEO3) as previously described (Rutkowski, D. T., et al. (2003) J. Biol. Chem. 278(32):30365-30372, Rutkowski D. T., et al. (2001) Proc. of the Nat. Acad. of Sci. of the U.S.A. 98:7823-7828), to determine if the organization of the translocon has changed in response to the swapped signal sequence, as observed in some cases (see appendix #3). In some but not all, cases this change has been observed to be independent of the r-mj (Rutkowski, D. T., et al. (2003) J. Biol. Chem. 278(32):30365-30372). Crosslinks are identified by co-immunoprecipitation or mass spectrometry analysis of bands isolated from SDS-PAGE (Schmitt-Ulms G., et al. (2001) J Mol Biol. 314(5): 1209-25). These studies provide a better understanding of the mechanism by which different conformers of pacp are obtained.

There is a molecular basis for the observation of conformer heterogeneity and its skew by swapping signal sequences. Either it will prove to be a variation on the themes observed in studies of other proteins (Rutkowski, D. T., et al. (2003) J. Biol. Chem. 278(32):30365-30372, Rutkowski D. T., et al. (2001) Proc. of the Nat. Acad. of Sci. of the U.S.A. 98:7823-7828), or it will be a new mechanism. In either case, its relevance to prostate cancer may be indicated by the nature of crosslinked proteins identified, in conjunction with the outcome of the experiments described above which relate conformers to prostate cancer pathogenesis indirectly or directly, respectively.

All publications and patent applications mentioned in this specification are indicative of the level if skill of those in the art to which this invention pertains. All publications and patent applications are herein incorporated by reference to the same extent as if each individual publication or patent application was specifically and individually indicated to be incorporated by reference.

The invention now having been fully described, it will be apparent to one of ordinary skill in the art than many changes and modifications can be made thereto without departing from the spirit or scope of the appended claims.

What is claimed is:

1. A method for identifying a one or more prostate disease-related conformer of prostatic acid phosphatase, said method comprising:
contacting a biological fluid sample from a patient with a disease of the prostate with a plurality of conformer-specific antibodies to prostatic acid phosphatase;
detecting substantially specific binding of said antibodies to prostatic acid phosphatase conformers in said sample as compared to a control sample as indicative of the presence of one or more disease-related conformer of prostatic acid phosphatase in said sample.

2. A method for identifying a molecule that inhibits a disease-related conformer of prostatic acid phosphatase, said method comprising:
contacting a molecule that binds to prostatic acid phosphatase with a disease-related conformer of prostatic acid phosphatase; and
evaluating whether said molecule inhibits said disease-related conformer of prostatic acid phosphatase, whereby a molecule that inhibits said disease related conformer is identified.

3. A method for identifying one or more antibodies that are substantially specific for a conformer of prostatic acid phosphatase, said method comprising:
contacting a plurality of conformers of prostatic acid phosphatase with a plurality of antibodies to conformers of prostatic acid phosphatase; and
evaluating the specificity of binding of said antibodies to individual members of said plurality of conformers of prostatic acid phosphatase, whereby one or more antibodies that are substantially specific are identified.

4. A method for establishing a prostatic acid phosphatase conformer profile in a population of individuals having a disease of the prostate, said method comprising:
compiling a prostatic acid phosphatase conformer profile of individual members of said population; and
establishing a relationship between prostatic acid phosphatase conformer profiles of said individual members and specific characteristics of said disease of the prostate in said individual members.

5. The method according to claim 4, wherein said specific characteristics of said disease include the response to treatment of said individual members.

6. The method according to claim 4, wherein said specific characteristics of said disease include the diagnoses of said individual members.

7. A method for bioconformatically selecting a treatment to administer to a patient having a disease of the prostate, said method comprising:
determining a prostatic acid phosphatase conformer profile of said patient; comparing said prostatic acid phosphatase conformer profile of said patient to a prostatic acid phosphatase conformer population profile obtained according to the method of claim 5; and
selecting as a method of treatment for said patient a method of treatment that was successful for treatment of individual members of said population having a substantially similar conformer profile.

8. A monoclonal antibody which is substantially specific for a conformer of prostatic acid phosphatase.

9. The monoclonal antibody according to claim 8, wherein said conformer is a disease related conformer.

10. The monoclonal antibody according to claim 9, wherein said disease is prostate cancer.

11. A cell line which produces a monoclonal antibody according to any one of claims 8 to 10.

12. A method for screening for the presence of prostate cancer in an individual, said method comprising:
contacting a fluid or cells of said individual with a monoclonal antibody according to claim 10 or binding fragment thereof, wherein said monoclonal antibody or binding fragment thereof is labelled with a detectable label; and
detecting said label as indicative of the presence of prostate cancer in said individual.

13. The method according to claim 12, wherein said detectable label is a radionuclide, a fluorescer, an enzyme or a chemiluminscer.

14. The method according to claim 10, wherein said cells or tissues are obtained from the patient and exposed to the monoclonal antibody in vitro under conditions conducive to immune complex formation.

15. A conformer of prostatic acid phosphatase, wherein said conformer is produced by the method of:
expressing in a fractionated lysate a prostatic acid phosphatase gene comprising a substituted signal sequence coding region, wherein said substituted signal sequence coding region is other than a native signal sequence coding region and is of a different class than said native signal sequence coding region, whereby a prostatic acid phosphatase having an altered conformation is produced.

16. A method for obtaining a conformer of a prostatic acid phosphatase, said method comprising:
expressing in a fractionated lysate a prostatic acid phosphatase gene comprising a substituted signal sequence coding region, wherein said substituted signal sequence coding region is other than a native signal sequence coding region and is of a different class than said native signal sequence coding region, whereby a prostatic acid phosphatase having an altered conformation is produced.

17. The method according to claim 16, wherein said substituted signal sequence coding region is a mutated signal sequence coding region.

18. A method for preparing antibodies to a conformer obtained according to the method of claim 16, said method comprising the step of:
immunizing a mammalian host with said conformer under conditions whereby antibodies are produced which bind substantially specifically to said conformer as compared to other conformers of said prostatic acid phosphatase.

19. The method according to claim 18, further comprising:
after said immunizing step, immortalizing B-lymphocytes from said mammalian host to provide immortalized B-lymphocytes secreting antibodies;
screening said antibodies for substantial specificity for said conformer whereby antibodies with substantial specificity are obtained; and
isolating immortalized B-lymphocytes secreting said antibodies with substantial specificity.

20. The method according to claim 19, wherein said mammalian host is a mouse, said B-lymphocytes are splenocytes and said immortalizing is with neoplastic cells to produce hybridomas.

21. The method according to claim 20, wherein said mouse is a prostatic acid phosphatase knockout mouse.

22. A pharmaceutical composition comprising:
a prostate disease-related conformer of prostatic acid phosphatase; and
a pharmaceutically acceptable adjuvant.

23. A method for treating a prostate disease, said method comprising the step of inoculating a host with a composition according to claim 22.

24. The method according to claim 23, wherein said prostate disease is prostatic cancer.
